(19) United States
(12) Patent Application Publication
(10) Pub. No.: US 2024/0238248 A1
(43) Pub. Date: Jul. 18, 2024

(54) PHARMACEUTICAL COMPOSITION FOR USE IN TREATING CEREBRAL INFARCTION

(71) Applicant: Ji Xing Pharmaceuticals Hong Kong Limited, Shanghai (HK)

(72) Inventors: Naoko NISHIMURA, Tokyo (JP); Keiji HASUMI, Tokyo (JP); Kazuo HONDA, Tokyo (JP)

(21) Appl. No.: 18/289,399

(22) PCT Filed: May 10, 2022
(86) PCT No.: PCT/US2022/028488

§ 371 Date: Nov. 3, 2023

(30) Foreign Application Priority Data
May 10, 2021 (JP) 2021-079820

(51) Int. Cl. A61K 31/4035; A61P 9/10

(52) U.S. Cl. CPC A61K 31/4035; A61P 9/10

(57) ABSTRACT

To provide a safe and effective therapy and pharmaceutical composition using SMTP-7 (Compound I) or a salt thereof in cerebral infarction patients. A pharmaceutical composition for treating cerebral infarction, comprising SMTP-7 (Compound I) or a pharmacological acceptable salt thereof, which is administered at a dose of 1 to 6 mg/kg as SMTP-7 (Compound I) or a salt thereof.

### CROSS-REFERENCE TO RELATED APPLICATIONS

This application claims priority to Japanese Patent Application Serial No. JP2021-079820, filed May 10, 2021, the disclosure of which is hereby incorporated herein by reference in its entirety for all purposes.

### TECHNICAL FIELD

The present invention relates to a pharmaceutical composition for treating cerebral infarction, comprising SMTP-7 with a specific dosage, as active ingredient.

### BACKGROUND ART

Cerebral infarction is a major cause of death in developed countries. Even if life is saved, residual disability such as paralysis is likely to remain. Cerebral infarction is definitively treated by recanalization of an obstructed site; however, resupply of blood flow to a site damaged by cerebral infarction may cause hemorrhagic cerebral infarction and worsen life and functional prognosis.

As a therapy for recanalization of occluded vessel employed in cerebral infarction acute-phase, thrombolytic therapy by intravenous administration of a thrombolytic agent, rt-PA, and mechanical thrombectomy by endovascular surgery, are known. The rt-PA, currently used as a first-choice drug can be administered to patients by 4.5 hours after onset. The agent has a strong thrombolytic effect but likely causes a hemorrhagic side effect. Other than application timing, strict restrictions are present. The percentage of allowable patients for administration is less than 10% of the whole cerebral infarction patients. Even if rt-PA is applicable, a few percent of the patients develop serious intracranial hemorrhagic side effect, and conversely, disease states may sometimes deteriorate.

Endovascular surgery can be carried out only in limited institutions and the applicable condition for the surgery is main artery occlusion. Eighty to ninety percent of the patients fail to satisfy the conditions and are outside the applicable range. They are likely to cause hemorrhage due to reperfusion abnormality with the passage of time, similarly to the patients treated with rt-PA. In the circumstances, if a medical agent having not only a thrombolytic effect but also an action to suppress intracranial hemorrhage is developed, the number of applicable subjects increases and side effects can be reduced. Such a medical agent will be an innovative therapeutic agent for cerebral infarction.

SMTP (Stachybotrys Microspora Triprenyl Phenol) compounds, which refer to a group of compounds having a triprenyl phenol skeleton and produced by filamentous fungi, are known to have a thrombolysis promoter effect and an angiogenesis inhibitory effect (Patent Literatures 1 to 3). SMTP compounds induce a conformational change of plasminogen and consequently promote thrombolysis. Because of this, induction of excessive plasmin production can be avoided and risk of developing a hemorrhagic side effect is lessened (Non Patent Literature 1). In addition, SMTP compounds inhibit soluble epoxy hydrolase (sEH) to thereby produce an anti-inflammatory activity (Non Patent Literature 2) and an antioxidant activity, bringing a cerebroprotective action. The inventors performed experiments using animal models and already confirmed that one of the SMTP compounds, SMTP-7, has an effect to treat cerebral infarction (Patent Literatures 4).

### SUMMARY OF INVENTION

### Technical Problem

An object of the present invention is to provide a safe and effective therapy and pharmaceutical composition using SMTP-7 (Compound I) or a salt thereof in cerebral infarction patients.

### Solution to Problem

The present inventors conducted clinical trials in which SMTP-7 (Compound I) was administered to healthy persons and cerebral infarction patients. As a result they found a safe and effective dosage of SMTP-7 to cerebral infarction patients.

The present invention is based on the results of the above clinical trial and relates to the following [1] to [50].
[1] A pharmaceutical composition for treating cerebral infarction, comprising Compound I shown in the following formula (I) or a salt thereof, which is administered to a subject at a dose of 1 to 6 mg/kg as said Compound I.

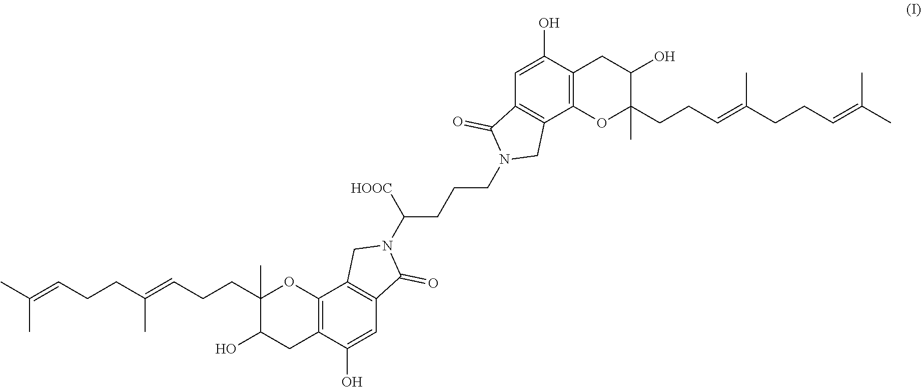 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

[2] The pharmaceutical composition according to [1], which is administered to a subject at a dose of 1 mg/kg as said Compound I.
[3] The pharmaceutical composition according to [1], which is administered to a subject at a dose of 3 mg/kg as said Compound I.
[4] The pharmaceutical composition according to [1], which is administered to a subject at a dose of 6 mg/kg as said Compound I.
[5] The pharmaceutical composition according to any one of [1] to [4], which is administered to a subject within 12 hours of the onset of cerebral infarction.
[6] The pharmaceutical composition according to any one of [1] to [4], which is administered to a subject 3 to 12 hours after the onset of cerebral infarction.
[7] The pharmaceutical composition according to any one of [1] to [4], which is administered to a subject 4.5 to 12 hours after the onset of cerebral infarction.
[8] The pharmaceutical composition according to any one of [1] to [7], which is administered to a subject having a risk of causing hemorrhage.
[9] The pharmaceutical composition according to [8], wherein the hemorrhaging is intracranial or extracranial hemorrhage, e.g., intracranial hemorrhage such as symptomatic intracranial hemorrhage or intracranial hemorrhage with NIHSS score worsening of 4 or more points.
[10] The pharmaceutical composition according to any one of [1] to [9], which is administered to a subject with cerebral infarction having vascular occlusion including partial occlusion.
[11] The pharmaceutical composition according to any one of [1] to [10], which is administered intravenously.
[12] The pharmaceutical composition according to any one of [1] to [11], which is administered once daily, preferably in a single dose.
[13] The pharmaceutical composition according to any one of [1] to [12], which is administered as a bolus followed by continuous administration.
[14] The pharmaceutical composition according to [13] which is administered intravenously over 1 minute followed by infusion over 30 minutes.
[15] The pharmaceutical composition according to any one of [1] to [14], which can be administered to a subject to whom administration of a thrombolytic agent or physical thrombectomy is difficult or impossible.
[16] The pharmaceutical composition according to [15], wherein the thrombolytic agent is a tissue plasminogen activator.
[17] The pharmaceutical composition according to any one of [1] to [16], wherein the cerebral infarction is atherothrombotic/embolic cerebral infarction, cardiogenic cerebral infarction or lacunar cerebral infarction.
[18] The pharmaceutical composition according to any one of [1] to [17], wherein a single dose of Compound I or a salt thereof is contained in a sealed container together with a pharmacologically acceptable carrier.
[19] The pharmaceutical composition according to any one of [1] to [18], wherein 20 mg to 1000 mg of Compound I or a salt thereof is contained in a sealed container together with a pharmacologically acceptable carrier.
[20] A pharmaceutical composition for treating cerebral infarction, comprising Compound I or a salt thereof, which is administered to a subject within 12 hours of the onset of cerebral infarction.
[21] The pharmaceutical composition according to [20], which is administered to a subject 3 to 12 hours after the onset of cerebral infarction.
[22] The pharmaceutical composition according to [20], which is administered to a subject 4.5 to 12 hours after the onset of cerebral infarction.
[23] The pharmaceutical composition according to any one of [20] to [22], which is administered to a subject at a dose of 1 to 6 mg/kg as said Compound I.
[24] The pharmaceutical composition according to any one of [20] to [22], which is administered to a subject at a dose of 1 mg/kg as said Compound I.
[25] The pharmaceutical composition according to any one of [20] to [22], which is administered to a subject at a dose of 3 mg/kg as said Compound I.
[26] The pharmaceutical composition according to any one of [20] to [22], which is administered to a subject at a dose of 6 mg/kg as said Compound I.
[27] The pharmaceutical composition according to any one of [20] to [26], which is administered to a subject having a risk of causing hemorrhage.
[28] The pharmaceutical composition according to [27], wherein the hemorrhage is intracranial or extracranial hemorrhage, e.g., intracranial hemorrhage such as symptomatic intracranial hemorrhage or intracranial hemorrhage with NIHSS score worsening of 4 or more points.
[29] The pharmaceutical composition according to any one of [20] to [28], which is administered to a subject with cerebral infarction having vascular occlusion including partial occlusion.
[30] The pharmaceutical composition according to any one of [20] to [29], which is administered intravenously.
[31] The pharmaceutical composition according to any one of [20] to [30], which is administered once daily, preferably in a single dose.
[32] The pharmaceutical composition according to any one of [20] to [31], which is administered as a bolus followed by continuous administration.
[33] The pharmaceutical composition according to [32] which is administered intravenously over 1 minute followed by infusion over 30 minutes.
[34] The pharmaceutical composition according to any one of [20] to [33], which can be administered to a subject to whom administration of a thrombolytic agent or physical thrombectomy is difficult or impossible.
[35] The pharmaceutical composition according to [34], wherein the thrombolytic agent is a tissue plasminogen activator.
[36] The pharmaceutical composition according to any one of [20] to [35], wherein the cerebral infarction is atherothrombotic/embolic cerebral infarction, cardiogenic cerebral infarction or lacunar cerebral infarction.
[37] A pharmaceutical composition for treating cerebral infarction, comprising Compound I or a salt thereof, wherein the composition is to recanalize occluded vessel in cerebral infarction (the composition is occluded vessel recanalizing agent).
[38] A pharmaceutical composition for treating cerebral infarction, comprising Compound I or a salt thereof, wherein the composition is administered to a subject having a risk of causing hemorrhage.
[39] A pharmaceutical composition for reducing risk of causing hemorrhage in cerebral infarction, comprising Compound I or a salt thereof.
[40] A pharmaceutical composition for treating cerebral infarction, comprising Compound I or a salt thereof, wherein the composition is to improve a life of independence after cerebral infarction.
[41] The pharmaceutical composition according to [40], in which an index of the improvement of a life of independence is outcome of mRS (modified Rankin Scale) of 0-1 on Day 90 after administration.
[42] The pharmaceutical composition according to [40], wherein an index of the improvement of a life of independence is outcome of mRS (modified Rankin Scale) of 0-2 on Day 90 after administration.
[43] A pharmaceutical composition for treating cerebral infarction, comprising Compound I or a salt thereof, wherein the composition has an action to dissolve a thrombus of a cerebral infarction patient.
[44] A pharmaceutical composition for treating cerebral infarction, comprising Compound I or a salt thereof, wherein the composition has a low risk of a hemorrhagic side effect. The risk of a hemorrhagic side effect is low in comparison with, for example, an existing cerebral infarction drug (for example, tPA).
[45] The pharmaceutical composition according to [44], wherein the hemorrhagic side effect is symptomatic intracranial hemorrhage.
[46] The pharmaceutical composition according to any one of [1] to [45], comprising either or both of a basic additive and an amphipathic additive.
[47] The pharmaceutical composition according to [46], wherein the basic additive is one or more selected from the group consisting of amino sugars, alkanolamines and trometamol salts, preferably, one or more selected from the group consisting of meglumine, triethanolamine and trometamol hydrochloride, and wherein the amphipathic additive is one or more selected from the group consisting of polyoxyethylene castor oil, polyoxyethylene hydrogenated castor oil, polyoxyethylene sorbitan monolaurate, polyoxyethylene-polyoxypropylene glycol, polysorbate, polyethylene glycol, ursodesocycholic acid, sorbitan fatty acid ester and sodium desoxycholate, preferably, one or more selected from the group consisting of polyoxyethylene castor oil, polyoxyethylene hydrogenated castor oil and polyoxyethylene sorbitan monolaurate.
[48] The pharmaceutical composition according to any one of [1] to [47], wherein Compound I is SMTP-7 represented by formula (II):

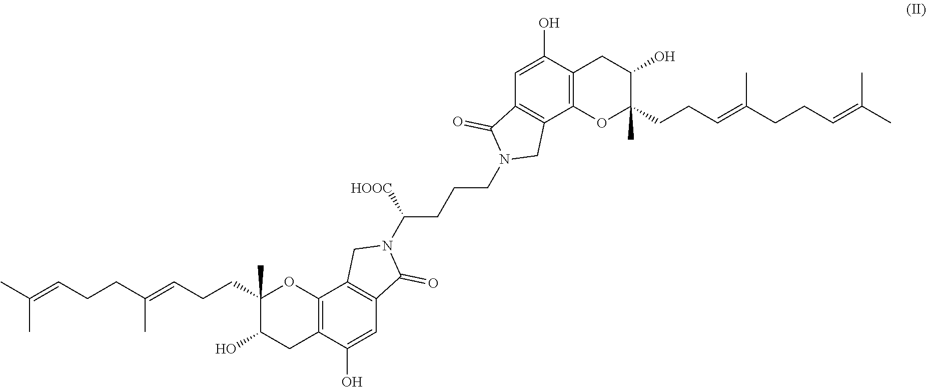 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

[49] The pharmaceutical composition according to any one of [1] to [48], wherein cerebral infarction is ischemic stroke.
[50] The pharmaceutical composition according to [49], wherein the ischemic stroke is acute ischemic stroke.

The pharmaceutical compositions according to [37] to [45] may have one or more characteristics defined by [1] to [19].

The present invention according to another aspect provides the following [1] to [43].
[1] A method for treating cerebral infarction, comprising administering Compound I shown in the following formula (I) or a salt thereof to a subject, wherein Compound I or a salt thereof is administered at a dose of 1 to 6 mg/kg as said Compound I.

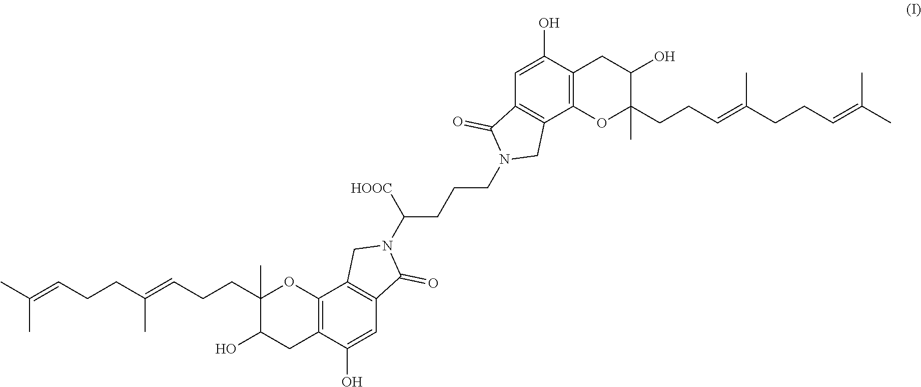 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

[2] The method according to [2], wherein Compound I or a salt thereof is administered to a subject at a dose of 1 mg/kg as said Compound I.
[3] The method according to [2], wherein Compound I or a salt thereof is administered to a subject at a dose of 3 mg/k as said Compound I.
[4] The method according to [2], wherein Compound I or a salt thereof is administered to a subject at a dose of 6 mg/kg as said Compound I.
[5] The method according to any one of [1] to [4], wherein the subject is a subject within 12 hours of the onset of cerebral infarction.
[6] The method according to any one of [1] to [4], wherein the subject is a subject 3 to 12 hours after the onset of cerebral infarction.
[7] The method according to any one of [1] to [4], wherein the subject is a subject 4.5 to 12 hours after the onset of cerebral infarction.
[8] The method according to any one of [1] to [7], wherein the subject is a subject having a risk of causing hemorrhage.
[9] The method according to [8], wherein the hemorrhage is intracranial or extracranial hemorrhage, e.g., intracranial hemorrhage such as symptomatic intracranial hemorrhage or intracranial hemorrhage with NIHSS score worsening of 4 or more points.
[10] The method according to any one of [1] to [9], wherein the subject has a subject having vascular occlusion including partial occlusion.
[11] The method according to any one of [1] to [10], wherein Compound I or a salt thereof is administered intravenously.
[12] The method according to any one of [1] to [11], wherein Compound I or a salt thereof is administered once daily, preferably in a single dose.
[13] The method according to any one of [1] to [12], wherein Compound I or a salt thereof is administered as a bolus followed by continuous administration.
[14] The method according to [13], wherein Compound I or a salt thereof is administered intravenously over 1 minute followed by infusion over 30 minutes.
[15] The method according to any one of [1] to [14], wherein the subject includes a subject to whom administration of a thrombolytic agent or physical thrombectomy is difficult or impossible.
[16] The method according to [15], wherein the thrombolytic agent is a tissue plasminogen activator.
[17] The method according to any one of [1] to [16], wherein the cerebral infarction is atherothrombotic/embolic cerebral infarction, cardiogenic cerebral infarction or lacunar cerebral infarction.
[18] A method for treating cerebral infarction, comprising administering Compound I or a salt thereof to a subject, wherein the subject is a subject within 12 hours of the onset of cerebral infarction.
[19] The method according to [18], wherein the subject is a subject 3 to 12 hours after the onset of cerebral infarction.
[20] The method according to [18], wherein the subject is a subject 4.5 to 12 hours after the onset of cerebral infarction.
[21] The method according to any one of [18] to [20], wherein Compound I or a salt thereof is administered to a subject at a dose of 1 to 6 mg/kg as said Compound I.
[22] The method according to any one of [18] to [20], wherein Compound I or a salt thereof is administered to a subject at a dose of 1 mg/kg as said Compound I.
[23] The method according to any one of [18] to [20], wherein Compound I or a salt thereof is administered to a subject at a dose of 3 mg/kg as said Compound I.
[24] The method according to any one of [18] to [20], wherein Compound I or a salt thereof is administered to a subject at a dose of 6 mg/kg as said Compound I.
[25] The method according to any one of [18] to [24], wherein the subject is a subject having a risk of causing hemorrhage.
[26] The method according to [25], wherein the hemorrhage is intracranial or extracranial hemorrhage, e.g., intracranial hemorrhage such as symptomatic intracranial hemorrhage or intracranial hemorrhage with NIHSS score worsening of 4 or more points.
[27] The method according to any one of [18] to [26], wherein the subject has vascular occlusion including partial occlusion.
[28] The method according to any one of [18] to [27], wherein Compound I or a salt thereof is administered intravenously.
[29] The method according to any one of [18] to [28], wherein Compound I or a salt thereof is administered once daily, preferably in a single dose.
[30] The method according to any one of [18] to [29], wherein Compound I or a salt thereof is administered as a bolus followed by continuous administration.
[31] The method according to [30], wherein Compound I or a salt thereof is administered intravenously over 1 minute followed by infusion over 30 minutes.
[32] The method according to any one of [18] to [31], wherein the subject includes a subject to whom administration of a thrombolytic agent or physical thrombectomy is difficult or impossible.
[33] The method according to [32], wherein the thrombolytic agent is a tissue plasminogen activator.
[34] The method according to any one of [18] to [33], wherein the cerebral infarction is atherothrombotic/embolic cerebral infarction, cardiogenic cerebral infarction or lacunar cerebral infarction.
[35] A method for recanalizing occluded vessel, comprising administering Compound I or a salt thereof to a subject.
[36] A method for treating cerebral infarction, comprising administering Compound I or a salt thereof to a subject, wherein the subject is a subject having a hemorrhagic risk.
[37] A method for reducing a hemorrhagic risk in cerebral infarction, comprising administering Compound I or a salt thereof to a subject.
[38] A method for improving a life of independence after cerebral infarction, comprising administering Compound I or a salt thereof to a subject. Alternatively, a method for treating cerebral infarction, comprising administering Compound I or a salt thereof to a subject, wherein a life of independence after cerebral infarction is improved.
[39] The method according to [38], wherein an index of the improvement of a life of independence is outcome of mRS (modified Rankin Scale) of 0-1 on Day 90 after administration.
[40] The method according to [38], wherein an index of the improvement of a life of independence is outcome of mRS (modified Rankin Scale) of 0-2 on Day 90 after administration.
[41] A method for dissolving a thrombus of a cerebral infarction patient, comprising administering Compound I or a salt thereof. Alternatively, a method for treating cerebral infarction, comprising administering Compound I or a salt thereof, wherein a thrombus of a cerebral infarction patient is dissolved.
[42] A method for treating cerebral infarction, comprising administering Compound I or a salt thereof to a subject, wherein risk of a hemorrhagic side effect is low. The risk of a hemorrhagic side effect is low in comparison with, for example, an existing cerebral infarction drug (for example, tPA).
[43] The method according to [42], wherein the hemorrhagic side effect is symptomatic intracranial hemorrhage.

The methods according to [35] to [43] may have one or more characteristics defined by [1] to [17].

### Advantageous Effects of Invention

According to the present invention, it is possible to treat cerebral infarction by use of safe and effective SMTP-7. In particular, cerebral infarction patients, to which existing thrombolytic agents cannot be applied, can be treated with a novel drug therapy.

### BRIEF DESCRIPTION OF DRAWINGS

FIG. 1 shows a flowchart of a placebo-control, randomized double-blind test (phase I test) for healthy persons.

FIG. 2 shows pharmacokinetic parameters of plasma TMS-007 compared in rats, monkeys and humans.

FIG. 3 shows pharmacokinetic parameters (plasma TMS-007 concentration, Cmax, AUC) in monkeys and humans.

FIG. 4 shows pharmacokinetic parameters in humans (healthy persons).

FIG. 5 shows details of subjects in a single dose study (phase II test) for cerebral infarction patients.

FIG. 6 shows details of analysis sets in a single dose study (phase II test) for cerebral infarction patients.

FIG. 7 shows mRS original score on Day 90 after administration between a placebo group and (TMS-007 1 mg-administration group) FAS group.

FIG. 8 shows mRS original score on Day 90 after administration between a placebo group and (TMS-007 3 mg-administration group) FAS group.

FIG. 9 shows mRS original score on Day 90 after administration between a placebo group and (TMS-007 6 mg-administration group) FAS group.

FIG. 10 shows mRS original score on Day 90 after administration between a placebo group and (TMS-007-combined group) FAS group.

FIG. 11 shows a box-and-whisker plot of plasma drug concentration in different dose groups 31 minutes after initiation of administration of a study drug, PK analysis set.

### DESCRIPTION OF EMBODIMENTS

In the specification, when the numerical range is expressed as A “to” B, the numerical values before and after “to” are included as the lower limit and the upper limit thereof, respectively.

In the specification, when the amount of a component of a composition is described, if a plurality of substances corresponding to the component are present in the composition, the amount refers to a total amount of the substances (present in the composition), unless otherwise specified.

In the specification, when a group (atomic group) is described, if none of the descriptions of “substituted” and “unsubstituted” are found a group having a substituent and a group having no substituent are both included. For example, an “alkyl group” includes not only an alkyl group having no substituent (unsubstituted alkyl group) but also an alkyl group having a substituent (substituted alkyl group).

In the specification, the term “step” includes not only an independent step but also an indistinguishable step as long as an intended work can be attained in the step, even if it cannot be clearly distinguished from another step.

In the specification, the “mass %” and “wt %” are interchangeably used, and “parts by mass” and “parts by weight” are interchangeably used. In the specification, the terms “weight average molecular weight (Mw)” and “number average molecular weight (Mn)” refer to, unless otherwise specified, molecular weights obtained by separating by a gel permeation chromatographic (GPC) apparatus using a column of TSKgel GMHxL, TSKgel G4000HxL or TSKgel G2000HxL (all are trade names, manufactured by Tohso Corporation) and a solvent THF (tetrahydrofuran), and detecting by a differential refractometer, through calculation based on polystyrene used as a standard substance.

### 1. Pharmaceutical Composition

### (1) Active Ingredient

The pharmaceutical composition according to the present invention contains Compound I represented by the formula (I), specifically comprising SMTP-7 represented by formula (II), as an active ingredient.

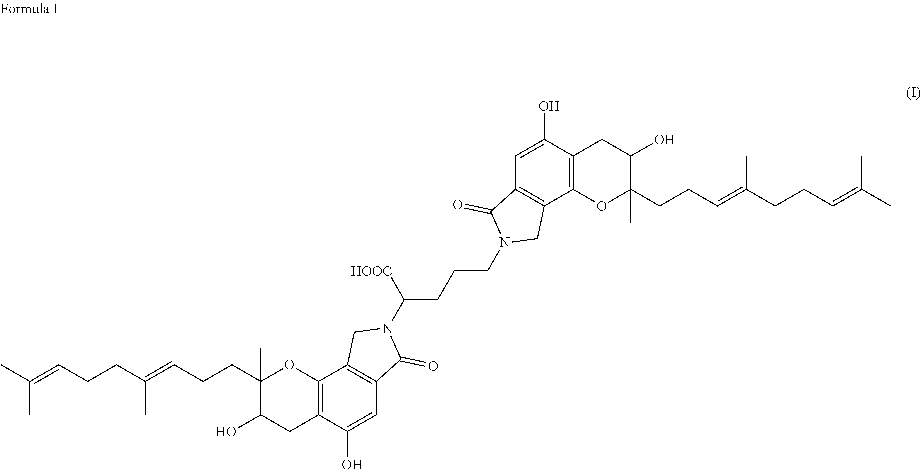 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

Chemical name: 2,5-bis-[8-(4,8-dimethyl-nona-3,7-dienyl)-5,7-dihydroxy8-methyl-3-oxo-3,6,7,8-tetrahydro-1H-9-oxa-2-azacyclopenta[a]naphthalen-2-yl]-pentanoic acid

C₅₁H₆₈N₂O₁₀ Molecular formula
Molecular weight: 869.09

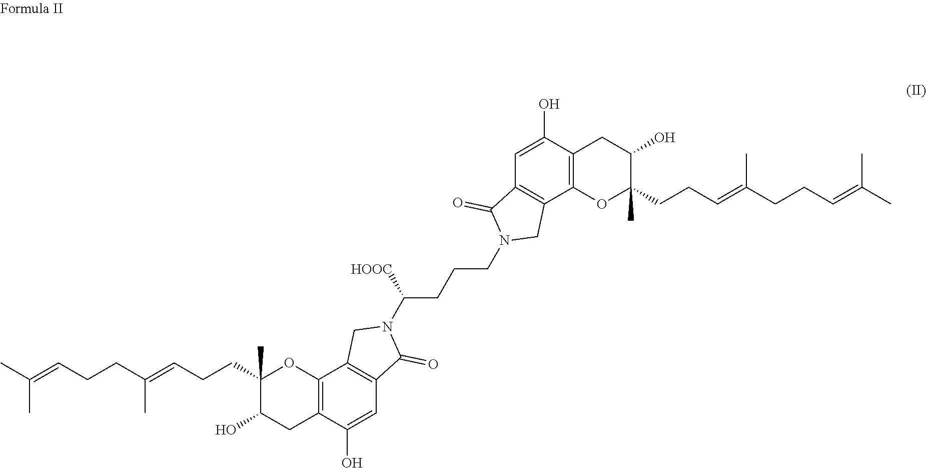 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

Chemical name: (S)-2,5-bis((2S,3S)-2-((E)-4,8-dimethylnona-3,7-dien-1-yl)-3,5-dihydroxy-2-methyl-7-oxo-3,4,7,9-tetrahydropyrano[2,3-e]isoindol-8(2H)-yl)pentanoic acid

C₅₁H₆₈N₂O₁₀ Molecular formula
Molecular weight: 868.49

Compound I may be present in the form of a pharmaceutical acceptable salt. An inorganic acid and an organic acid such as hydrochloric acid, hydrogen bromide, sulfuric acid, nitric acid, phosphoric acid, or citric acid; and formic acid, fumaric acid, malic acid, acetic acid, succinic acid, tartaric acid, methanesulfonic acid or p-toluenesulfonic acid; an alkali metal and an alkaline earth metal such as sodium, potassium, calcium or magnesium; a basic amine and a basic amino acid are suitably used for forming a salt of Compound I.

Examples of a suitable salt of Compound I include a sodium salt, a potassium salt, a calcium salt and a magnesium salt. A sodium salt and a potassium salt are preferable, and a sodium salt is most preferable.

Compound I may be chemically synthesized or obtained from a culture of a filamentous fungus such as Stachybotrys microspore through purification. A method for producing Compound I is described, for example, in Japanese Patent Publication No. 2004-224737, Japanese Patent Publication No. 2004-224738, and International Publication No. WO 2007/111203.

Compound I may be an enantiomer, a diastereomer, a mixture of enantiomers or a mixture of diastereomers. Such an enantiomer, a diastereomer, a mixture of enantiomers or a mixture of diastereomers may be obtained by chemical synthesis or from a culture of a filamentous fungus through purification. When Compound I is obtained from a filamentous fungus, it can be predominantly obtained by adding L-ornithine to a culture medium of the filamentous fungus.

Compound I has a thrombolytic effect, an anti-inflammatory effect and an antioxidant effect. As a result that Compound I exerts a cell protective effect and a neuroprotective effect in addition to the thrombolytic effect, and produces a preventive or therapeutic effect against ischemic damage such as cerebral infarction.

### (2) Composition/Dosage Form

In the pharmaceutical composition of the present invention, the content of Compound I relative to the total mass of the pharmaceutical composition is preferably 1 mass % to 80 mass %, more preferably 5 mass % to 60 mass % and further preferably 5 mass % to 50 mass %.

The pharmaceutical composition of the present invention is an intravenous preparation (injection). The intravenous preparation contains an aseptic aqueous or non-aqueous solvent, a suspension or an emulsifying agent. Examples of the aqueous solvent include distilled water for injection and saline. Examples of the non-aqueous solvent include alcohols such as ethanol. The pharmaceutical composition of the present invention may further contain a pharmaceutically acceptable carrier such as a tonicity agent, a preservative, a wetting agent, an emulsifier, a dispersant, a stabilizer or a dissolution aid.

Preferably, the pharmaceutical composition of the present invention contains basic additives and/or amphiphilic additives.

Examples of the basic additives include alkanolamines such as triethanolamine, monoethanolamine, diisopropanolamine and triisopropanolamine; trometamol (trishydroxymethylaminomethane) or a salt thereof; amino acids such as glycine; and amino sugars such as meglumine. Of them, at least one selected from the group consisting of an amino sugar, an alkanolamine and a trometamol salt is preferably contained; at least one selected from the group consisting of meglumine, triethanolamine and trometamol hydrochloride is more preferably contained; at least one of meglumine and trometamol hydrochloride is further preferably contained; and meglumine is further preferably contained.

The content of the basic additive relative to the total mass of the pharmaceutical composition is preferably 0.1 mass % to 60 mass %, more preferably 1 mass % to 40 mass %, and further preferably 2 mass % to 30 mass %.

As the amphiphile additive that can be used may be any one of an anionic amphipathic additive, a cationic amphipathic additive, an amphoteric amphipathic additive and a nonionic amphipathic additive. Of them, a nonionic amphipathic additive is preferable.

As the nonionic amphipathic additive, at least one selected from the group consisting of polyoxyethylene castor oil, polyoxyethylene hydrogenated castor oil, polyoxyethylene sorbitan monolaurate, polyoxyethylene-polyoxypropylene glycol, polysorbate, polyethylene glycol, ursodes oxycholic acid, sorbitan fatty acid ester, and sodium desoxycholate is preferable; at least one selected from the group consisting of polyoxyethylene castor oil, polyoxyethylene hydrogenated castor oil and polyoxyethylene sorbitan monolaurate, polyoxyethylene castor oil or polyoxyethylene hydrogenated castor oil is more preferable; and polyoxyethylene hydrogenated castor oil is particularly preferable.

The total number of oxyethylene units contained in the polyoxyethylene hydrogenated castor oil, although it is not particularly limited, is preferably 2 to 150, and more preferably 10 to 100.

If an amphiphile additive as mentioned above is contained, the content of the amphiphile additive relative to the total mass of the pharmaceutical composition is preferably 1 mass % to 80 mass %, more preferably 5 mass % to 70 mass %, and further preferably, 5 mass % to 60 mass %.

A preferable pharmaceutical composition of the present invention contains a sodium salt of Compound I, meglumine and polyoxyethylene hydrogenated castor oil.

The pharmaceutical composition of the present invention may contain an aseptic aqueous or non-aqueous solvent. Examples of the aqueous solvent include distilled water for injection and saline. Examples of the non-aqueous solvent include alcohols such as ethanol. Other than these, the pharmaceutical composition of the present invention may further contain a pharmacologically acceptable carrier such as a tonicity agent, a preservative, a wetting agent, an emulsifier, a dispersant, a stabilizer, a pH regulator (e.g., hydrochloric acid, sodium hydroxide) or a dissolution aid.

If the pharmaceutical composition contains other additives, the content of the other additives relative to the total mass of the pharmaceutical composition, although it varies depending on the dosage form of the pharmaceutical composition, is preferably 0.1 mass % to 80 mass %, and more preferably 1 mass % to 60 mass %. For example, in the case of a lyophilized injection formulation, which is prepared when used, the content of the other additives relative to the total mass of the pharmaceutical composition (except the mass of an injection for dilution) is preferably 0.1 mass to 80 mass %, and more preferably 1 mass to 60 mass %.

The pharmaceutical composition of the present invention may be a formulation prepared when used or a ready-to-use formulation. For example, the pharmaceutical composition may be a formulation obtained by putting Compound I or a salt thereof in an ampoule or a syringe together with a pharmacologically acceptable carrier and sealing it airtight.

The pharmaceutical composition of the present invention may contain an aseptic aqueous or non-aqueous solvent in addition to a basic additive and an amphiphile additive as mentioned above. Examples of the aqueous solvent include distilled water for injection and saline. Examples of the non-aqueous solvent include alcohols such as ethanol. Other than these, the pharmaceutical composition of the present invention may further contain a pharmacologically acceptable carrier, such as a tonicity agent, a preservative, a wetting agent, an emulsifier, a dispersant, a stabilizer or a dissolution aid.

### 2. Subject (Efficacy/Effect)

The pharmaceutical composition of the present invention is used for prevention or treatment of cerebral infarction, particularly treatment for cerebral infarction.

Cerebral infarction (including cerebral thrombosis and cerebral embolism), which is a target of the pharmaceutical composition of the present invention, include cerebral infarction within 12 hours after onset.

The pharmaceutical composition of the present invention can be applied to a subject to whom an existing reperfusion therapy is not easily applied or cannot be applied. Examples of the existing reperfusion therapy include administration of a thrombolytic drug and physical thrombectomy (percutaneous transluminal cerebral thrombectomy).

Examples of the thrombolytic agent include urokinase, pro-urokinase, tissue plasminogen activator (t-PA), nasaruplase and streptokinase. The t-PA may be a naturally occurring t-PA or a recombinant t-PA. The t-PAs currently clinical available are all recombinant t-PAs (rt-PA) such as alteplase, tisokinase, pamiteplase, and rt-PAs currently under development such as tenecteplase and desmoteplase.

If a thrombolytic agent is applied to cerebral infarction, the administration period of the thrombolytic agent is limited. For example, urokinase must be applied within 6 hours from onset and t-PA within 4.5 hours (in e.g., Japan) or within 3 hours (in e.g. USA) from onset. The pharmaceutical composition of the present invention, since the administration period is long, can be suitably applied to patients to which existing thrombolytic agents are not applicable, for example, patients within 3 to 12 hours or 4.5 to 12 hours after the onset of cerebral infarction. Note that, when the onset time is not clearly known, the latest symptomless time (the latest time when a patient was confirmed as symptomless) is deemed as the onset time.

The pharmaceutical composition of the present invention can be applied to a patient to which administration of a thrombolytic drug is cancelled due to contraindicated signs or symptoms during administration thereof. Examples of the contraindicated signs include hemorrhage diathesis, hemorrhage, hypertension, and impaired blood glucose. Alternatively, the pharmaceutical composition of the present invention can be applied also to a patient with cerebral infarction to which a thrombolytic drug cannot be administered because of a great risk of intracranial hemorrhage.

The pharmaceutical composition of the present invention can be applied before and after thrombectomy using a percutaneous transluminal cerebral thrombectomy device.

Cerebral infarctions are roughly classified based on causes into lacunar cerebral infarction, cardiogenic cerebral infarction and atherothrombotic cerebral infarction (atherothrombotic cerebral infarction and atheroembolic cerebral infarction). The pharmaceutical composition of the present invention may be applied to any one of the cerebral infarctions. Cerebral infarction is used interchangeably with ischemic stroke. Acute cerebral infarction is used interchangeably with acute ischemic stroke. Ischemic stroke is commonly acute ischemic stroke, which accounts for almost 90% of all strokes.

### 3. Dosage and Administration

The dose of the pharmaceutical composition of the present invention, although it is not particularly limited, preferably falls within the range of 1 to 6 mg/kg for adult, in Compound I (free form) equivalent. The dose is not limited as long as it falls within the range of 1 to 6 mg/kg. The dose may be any one of 1 to 3 mg/kg, 3 to 6 mg/kg, 1 mg/kg, 3 mg/kg and 6 mg/kg.

The frequency of administration, although it is not particularly limited, may be once or a plurality of times per day. The interval and period of the multiple administration can be selected by those skilled in the art in accordance with clinical findings, imaging findings, hematological findings, comorbidities and medical history.

The route of administration of the pharmaceutical composition of the present invention, although it is not particularly limited, is preferably intravenous administration.

The pharmaceutical composition of the present invention, more specifically, a single dose of Compound I or a salt thereof can be contained in a sealed container together with a pharmacologically acceptable carrier. The single dose can be obtained by multiplying the dose per body weight (1 kg) by the body weight of the patient. Commonly, a single dose can be obtained by multiplying the dose (for example, 1 to 6 mg/kg) per body weight (1 kg) by an adult average weight (for example, 50 kg, 60 kg, 70 kg). For example, if the dose per body weight (1 kg) is 1 to 6 mg/kg and an adult average weight is 60 kg, the single dose becomes 60 to 360 mg (in Compound I (free form) equivalent).

In the pharmaceutical composition of the present invention, Compound I or a salt thereof may be contained in a sealed container in an amount larger by 10%, 20%, 30% than the single dose (standard dose) so as to enable flexible control, together with a pharmacologically acceptable carrier. Alternatively, a ½ dose of Compound I or a salt thereof may be contained in a sealed container together with a pharmacologically acceptable carrier. In other words, in the pharmaceutical composition of the present invention, 20 mg to 1000 mg of Compound I or a salt thereof can be contained in a sealed container together with a pharmacologically acceptable carrier; preferably, 30 to 900 mg and more preferably 40 to 800 mg of Compound I or a salt thereof can be contained in a sealed container together with a pharmacologically acceptable carrier.

In a method for administrating the pharmaceutical composition of the present invention, although it is not limited, a single dose of Compound I is administered first as a bolus followed by continuous administration. For example, a method of administering 10% of a single dose as a bolus, followed by administering 90% thereof by infusion over 30 minutes to 1 hour, can be employed. Usually, intravenous administration is carried out over 1 minute followed by infusion over 30 minutes.

In the case of repeated administration, administration is preferably made immediately after onset, within 12 hours from onset and once daily for 7 days. Intravenous administration over 1 minute per time followed by infusion over 30 minutes is preferable.

The pharmaceutical composition of the present invention may be used in combination with another medical agent if it is not against the object of the invention. As described above, the pharmaceutical composition of the present invention can be administered to patients to which a thrombolytic agent is not applicable. However, combination use with a thrombolytic agent is not meant to be prohibited herein. Accordingly, the pharmaceutical composition of the present invention may be used in combination with a thrombolytic drug, a platelet aggregation inhibitor and a blood coagulation inhibitor (anticoagulant) as mentioned above.

After the onset of cerebral infarction, some of the patients are likely to become hemorrhagic due to damage to the blood vessel (for example, during reperfusion). However, the pharmaceutical composition of the present invention effectively prevents transition towards the hemorrhagic state and reduces hemorrhage tendency.

The pharmaceutical composition of the present invention reduces hemorrhagic risk compared to conventional cerebral infarction drugs or therapies (for example, tPA). Particularly, a risk of intracranial hemorrhage such as symptomatic intracranial hemorrhage is lower than existing therapies. The clinical phase II test (described later) suggests that symptomatic intracranial hemorrhage with NIHSS score worsening of 4 or more points.

A fundamentally treatment for cerebral infarction is recanalization of an occlusion site; however, the resupply of blood flow to a cerebral infarction site previously formed may induce hemorrhagic cerebral infarction to worsen life/functional prognosis. This is a dilemma case. The first-choice drug, tPA, for use in treatment of recanalization of occluded vessel in the acute stage of cerebral infarction has a strong thrombolytic effect but it is likely to produce a hemorrhagic side effect. The pharmaceutical composition of the present invention has a low risk of causing a hemorrhagic side effect (for example, symptomatic intracranial hemorrhage), compared to existing cerebral infarction drugs (for example, tPA). Since the pharmaceutical composition of the present invention has a thrombolytic ability, occluded vessel is recanalized and symptoms of a cerebral infarction patient are improved. Accordingly, the pharmaceutical composition of the present invention can be administered to a subject having hemorrhagic risk such as intracranial or extracranial hemorrhage and a subject with cerebral infarction having vascular occlusion including partial occlusion.

### 3. Method for Treating or Preventing Cerebral Infarction

The present invention provides a method for treating or preventing cerebral infarction comprising administering the pharmaceutical composition of the present invention or Compound I or a salt thereof to a patient in need. The dose of the pharmaceutical composition (Compound I or a salt thereof), interval, period and method of administration are the same as illustrated in the description of the pharmaceutical composition of the present invention. The method is characterized in that there is a low risk to cause hemorrhage in a subject and that occluded vessel including partial occlusion is recanalized.

The present invention also provides a method for recanalizing occluded vessel of cerebral infarction, comprising administering the pharmaceutical composition of the present invention or Compound I or a salt thereof to a patient in need thereof.

The present invention also provides a method for improving a life of independence after cerebral infarction, comprising administering the pharmaceutical composition of the present invention or Compound I or a salt thereof to a patient in need. The improvement of a life of independence can be evaluated based on, for example, outcome of mRS (modified Rankin Scale) 0-1 on Day 90 after administration or outcome of mRS (modified Rankin Scale) 0-2 on Day 90 after administration.

Cerebral infarction is improved by administration of the pharmaceutical composition of the present invention. Further, at least one selected from the group consisting of neurological symptoms associated with cerebral infarction, daily life performance impairment associated with cerebral infarction, and dysfunction associated with cerebral infarction can be improved.

### EXAMPLES

The present invention will be more specifically described by way of Examples but the present invention is not limited to these Examples. Note that, unless otherwise specified, “%” represents mass %.

### Reference Example 1: Toxicity Test by Two-Week Repeated Administration to Rats

Rats (12 males and 12 females) under neither anesthesia nor unrestrained condition were repeatedly and intravenously given TMS-007 (preparation of a sodium-salt thereof) at a dose of 2.4, 12 and 60 mg/kg (continuously over 30 minutes) once daily for two weeks. The day after completion of repeated administration, the rats were subjected to autopsy. As a result, NOAEL of the toxicity test of rats by repeated administration was determined to be 60 mg/kg or more.

### Reference Example 2: Toxicity Test by Two-Week Tepeated Administration to Cynomolgus Monkeys

Cynomolgus monkey (3 males per group and 3 females per group) under neither anesthesia nor unrestrained condition were repeatedly and intravenously given TMS-007 (preparation of a sodium-salt thereof) at a dose of 3, 10 and 30 mg/kg (continuously over 30 minutes) once daily for two weeks. The day after completion of repeated administration, the monkeys were subjected to autopsy. As a result, NOAEL of the toxicity test of cynomolgus monkeys by repeated administration was determined to be 10 mg/kg or more in both male and female groups.

### Example 1: Placebo-Control Randomized Double-Blind Test (Phase I Test) for Healthy Persons

### 1. Test Method

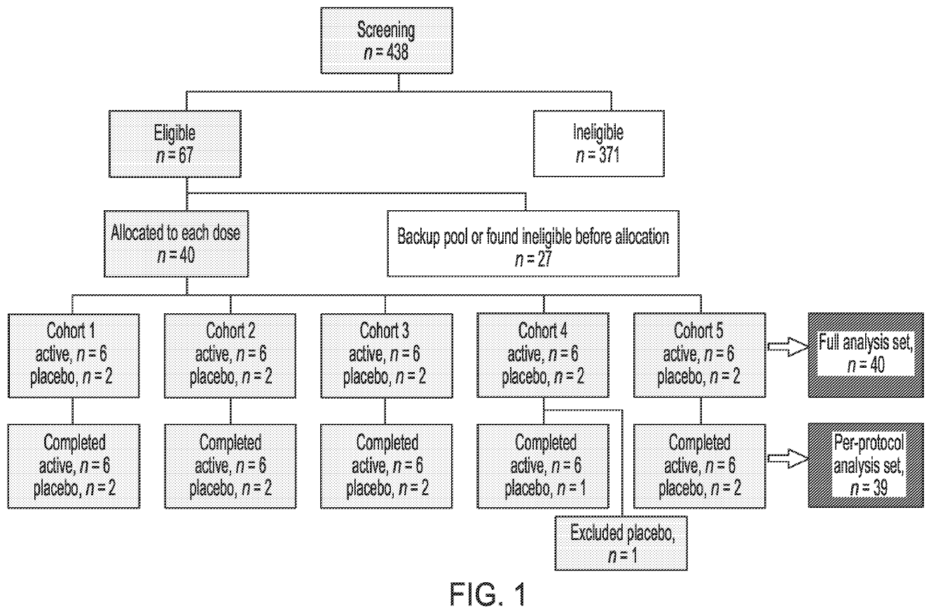

Phase I test (placebo-control randomized double-blind test) for healthy adult males was carried out. Five cohorts each was constituted of 8 persons (active drug was administered to 6 person and a placebo was administered to 2 person) were used in the test. TMS-007 (SMTP-7 sodium salt preparation) or a placebo was administered intravenously once at a dose of 0.05, 0.25, 1, 3, and 6 mg/kg (equivalent) over 31 minutes (bolus over 1 minute+continuous administration over 30 minutes). The subjects determined to be eligible by a screening test were hospitalized the day before administration. Day 3 from completion of administration, the subjects were allow to leave the hospital. Day 7 from completion of administration, post (follow-up) examination was carried out for safety, tolerance and PK in the period up to Day 7. The flowchart of the test is shown in FIG. 1.

### 2. Results

Adverse events observed were all non-serious/mild/restorability.

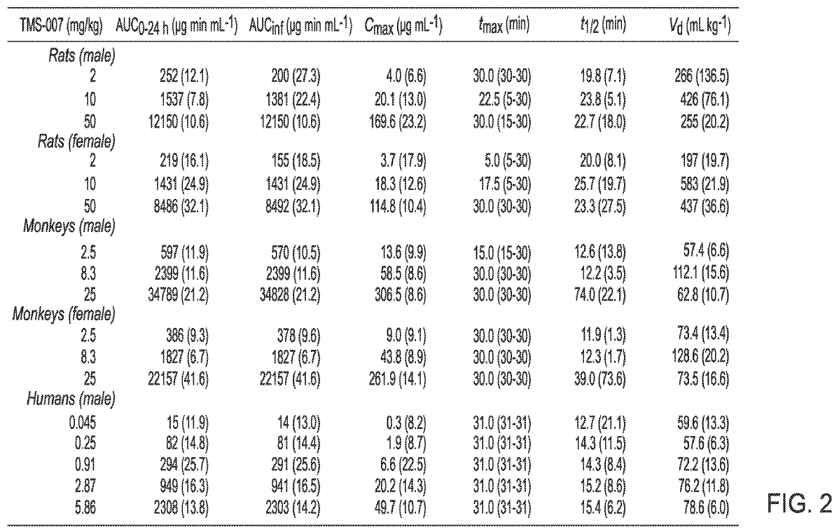

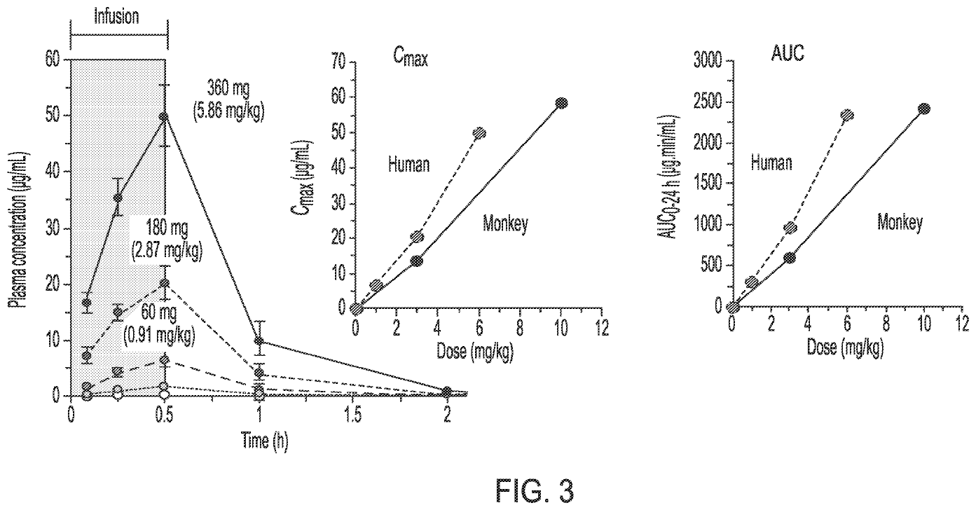

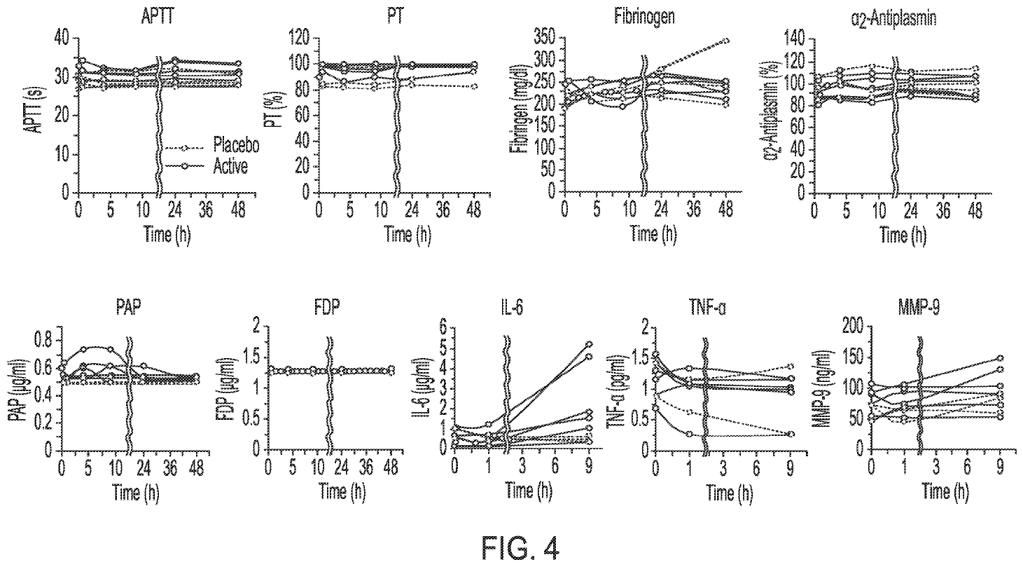

Cmax values of individual cohorts (administration of 0.05, 0.25, 1, 3, and 6 mg/kg) were 0.346 0.026, 1.86±0.15, 6.70 1.44, 20.3 2.8 and 49.9±5.2 μg/mL, respectively. AUC0-24 values of the individual cohorts were 15.1±1.7, 83.1±11.7, 301±73, 959±154 and 2330±330 μg-min/mL, respectively. The blood concentrations of TMS-007 all sharply increased after administration, reached a peak at 31 minutes immediately after completion of administration, and then, TMS-007 rapidly disappeared from the blood. The half-lives of TMS-007 in blood, were all about 10 to 15 minutes regardless of the dose. Unchanged form of TMS-007 in urine was not observed in any one of the cohorts. Pharmacokinetics (PK) data are shown in FIGS. 2 and 3, whereas pharmacodynamics (PD) data are shown in FIG. 4.

As mentioned above, when TMS-007 was administered intravenously once in a dose-escalation trial, indication of hemorrhagic risk was not observed in healthy persons. Satisfactory results were obtained with respect to safety, tolerance and others. Appreciable problems were not found in PK and PD profiles.

### 3. Consideration

In pharmacological tests using, e.g., mice, rats and monkeys, TMS-007 was already found to have medicinal effects such as a reduction of the area of infarction and improvement of neurological symptoms at a dose of 1 to 10 mg/kg. Cmax and AUC0-24 values when TMS-007 was administered to rats and monkeys were as follows. In the rats, Cmax and AUC0-24 values were 20.2 μg/mL and 1540 μg-min/mL, respectively (average value of a 12 mg/kg-dose group on the initial day of a repeated-dose study in male rats). In the monkeys, Cmax and AUC0-24 values were 45.8-58.6 μg/mL and 1930-2410 μg-min/mL, respectively (average value of a 10 mg/kg-dose group of an extended single-dose study and a repeated-dose study (the first day) in male monkeys).

From the above, it is expected that the medicinal effects of TMS-007 in humans can be obtained at a dose of 1 to 6 mg/kg. In a phase II test, the dose was set at 1, 3, and 6 mg/kg based on the safety results mentioned above and anticipated therapeutic dose.

### Example 2: Single Dose Study (Phase II Test) for Cerebral Infarction Patients

### 1. Test Method

### (1) Clinical Trial Design

Randomized, double blind, placebo control, single dose, intravenous administration, parallel between groups and dose escalation regimen

### (2) Subject

Cerebral infarction patients within 12 hours from onset are used as subjects. Test subjects (90 patients: first cohort: 9, second cohort: 27, third cohort: 54) are determined in accordance with the following selection criteria and exclusion criteria.

### Selection Criteria:

1) Persons who received adequate explanation in participating in this clinical trial and thereafter provided written informed consent (including written informed consent by a substitute)
2) Japanese males of 20 or more and less than 88 years old and Japanese females of 60 or more and less than 88 years old, who have symptomatic cerebral infarction
3) Persons having a body weight of 30 kg or more
4) Persons who can receive a study drug within 12 hours after the onset time confirmed or the latest symptomless time of cerebral infarction
5) Persons determined to have an NIHSS score of 6 or more and 23 or less at the time of examination for enrollment
6) Persons determined to have an MRI (DWI)-ASPECTS value (11 point method) of 5 or more at the time of examination for enrollment

### Exclusion Criteria:

### Clinical Symptom Clinical Test:

1) Persons determined by a doctor as being proper for receiving administration of rt-PA or endovascular treatment
2) Hemorrhage:
   a. Persons who are breeding (intracranial hemorrhage, gastrointestinal hemorrhage, urinary hemorrhage, retroperitoneal hemorrhage, hemoptysis)
   b. Persons suspected to have subarachnoid hemorrhage
   c. Persons having a platelet count of 100,000/mm³ or less before administration
   d. Persons having a prothrombin time-international normalized ratio (PT-INR) of 2.6 or more before administration
3) Blood pressure:
   a. Persons having a systolic blood pressure of 185 mmHg or more or a diastolic blood pressure of 110 mmHg or more, even if an appropriate antihypertensive treatment is applied before administration
4) Blood glucose:
   a. Persons having a blood glucose level of less than 50 mg/dL or beyond 400 mg/dL before administration
5) Pathology:
   a. Persons having wide-range early ischemic changes (absorption value of brain parenchyma slightly decreased or sulcus disappeared) observed by MRI before administration
   b. Persons having a displacement such as median plane deviation observed by MRI before administration
   c. Persons having seizure suspected as epilepsy at onset
   d. Patients having mild cerebral infarction symptoms or persons having major cerebral infarction symptoms rapidly improved
   e. Persons having mRS of 2 or more before onset
   f. In the third cohort, persons suspected by a doctor to have acute complete occlusion in internal carotid artery based on clinical and image data obtained at the examination for enrollment

### Disease Status, Medical History, Pregnancy Status:

6) Persons having a history of intracranial hemorrhage within a month before administration of a study drug, or having intracranial tumor, cerebral arteriovenous malformation, cerebral aneurysm or aortic dissection at the time of administration of a study drug
7) Persons having a history of an intracranial or spinal cord surgery or injury, or previous cerebral infarction within a month before administration of a study drug,
8) Persons having a history of gastrointestinal hemorrhage or urinary hemorrhage within 14 days before administration of a study drug
9) Persons received a major surgery within 7 day before administration of a study drug
10) Persons having a serious liver disorder
11) Persons having acute pancreatitis
12) Persons having a previous serious trauma within 7 day before administration of a study drug
13) Persons also having acute myocardial infarction or pericarditis after myocardial infarction
14) Persons having a serious malignant neoplasm
15) Persons pregnant (if a person suspected to be pregnant, a pregnancy test based on hCG in the blood or in urine is carried out)

### Allergy and Adverse Drug Reaction:

16) Persons having clinically important drug hypersensitivity or a history of hypersensitivity to a component of the drug to be tested

### Luxury Items/Medication History:

17) Persons who have participated in another clinical trial within three months and received a study drug (including a placebo)

### Others:

18) Persons do not provide informed consent (including informed consent to pregnancy avoidance precautions)
19) Persons having no health insurance
20) Persons to which a follow-up examination is not available
21) Persons to which MRI scan cannot be applied for the reasons of use of a pacemaker, an aneurysm clip or an implant device, claustrophobia or others
22) Persons determined as being unsuitable by, e.g., a chief doctor responsible for a trial

### (3) Studied Drugs

Test drug (TMS-007 (SMTP-7 sodium salt preparation))
Control drug (placebo)

### (4) Administration Schedule

To patients with acute cerebral infarction within 12 hours after onset, TMS-007 (SMTP-7 sodium salt preparation)) or a placebo were administered intravenously once (bolus over 1 minute+continuous injection over 30 minutes). The ratio of number of patients to which an active drug (TMS-007 sodium salt preparation) is to be administered relative to the number of patients to which a placebo is to be administered is set at 2:1 in the first and second cohorts and 1:1 in the third cohort as shown in Table 1 below.

TABLE 1
Administration of TMS-007 (SMTP-7) and placebo to patients.

| | | | Desired case number | |
|---|---|---|---|---|
| Cohort | Dose (in free form equivalent) | | TMS-007 | Placebo |
| 1 | TMS-007 (sodium salt prep- | 1 mg/kg | 6 | 3 |
| | aration) or placebo | | | |
| 2 | TMS-007 (sodium salt prep- | 3 mg/kg | 18 | 9 |
| | aration) or placebo | | | |
| 3* | TMS-007 (sodium salt prep- | 6 mg/kg | 27 | 27 |
| | aration) or placebo | | | |

*In the third cohort, the number of cases suspected of having lacunar infarction is set at 50% or less of the total cases of the third cohort (27 cases or less relative to the total 54 cases of the third cohort)

### (5) Dose Escalation and Discontinuation Criteria

The items for determining dose escalation are safety items, NIHSS, brain MRI, vital signs and, If necessary, PK data of all subjects in individual cohorts up to Day 7 after administration of a study drug. If some problems were found in safety items, whether the dose in the next cohort is reduced or not or transition to the next cohort is cancelled or not is considered. The degree of reduction of the dose is basically a 20% of the estimated dose of the next cohort, and can be up to 50%.

If any one of the following situations is satisfied, transition to the next cohort scheduled is cancelled. If necessary, change of dose may be considered.
1) Case where 4 patients or more died in cohort 1, 8 patients or more in cohort 2 and 16 patients or more in cohort 3, regardless of the presence or absence of causal relationship with a study drug
2) Case where the frequency of occurrence of symptomatic intracranial hemorrhage with NIHSS score worsening of 4 or more points within 24 hours is 4 or more in cohort 1, 11 or more in cohort 2, and 22 or more in cohort 3, regardless of the presence or absence of causal relationship with a study drug
3) case where discontinuation recommendation is issued from an independent data monitoring committee

Note that, elderly people of 86 or more years old are further evaluated for safety.

### (6) Endpoint

### Primary Endpoint

The frequency of occurrence of symptomatic intracranial hemorrhage with NIHSS score worsening of 4 or more points up to 24 hours after administration of TMS-007

### Secondary Endpoint

### Overall Mortality During the Test Period

Safety (e.g., extracranial hemorrhage, major hemorrhage) up to Day 7 after administration
Symptomatic intracranial hemorrhage and non-symptomatic intracranial hemorrhage within 24 hours after administration
Value of mRS on Day 90 after administration
A rate of recanalization observed in an MRA image of 24 hours after administration
PK (concentration of unchanged form of TMS-007, Cmax, AUC) when TMS-007 was administered

### Others

### (7) Analysis Set

### Safety Analysis Set: Subjects Receiving a Study Drug

Effectiveness analysis set: FAS is regarded as a main analysis set and sensitivity is analyzed based on analysis of PPS sets
FAS (full analysis set) set: subjects receiving a study drug and having effectiveness evaluation data defined in a clinical trial protocol
PPS (per protocol set) set: subjects of the FAS, who do not deviate from a clinical trial protocol
PK analysis set: subjects appropriately receiving a study drug and appropriately measured for TMS-007 concentration in plasma.
PD analysis set: subjects appropriately receiving a study drug and appropriately measured for blood markers

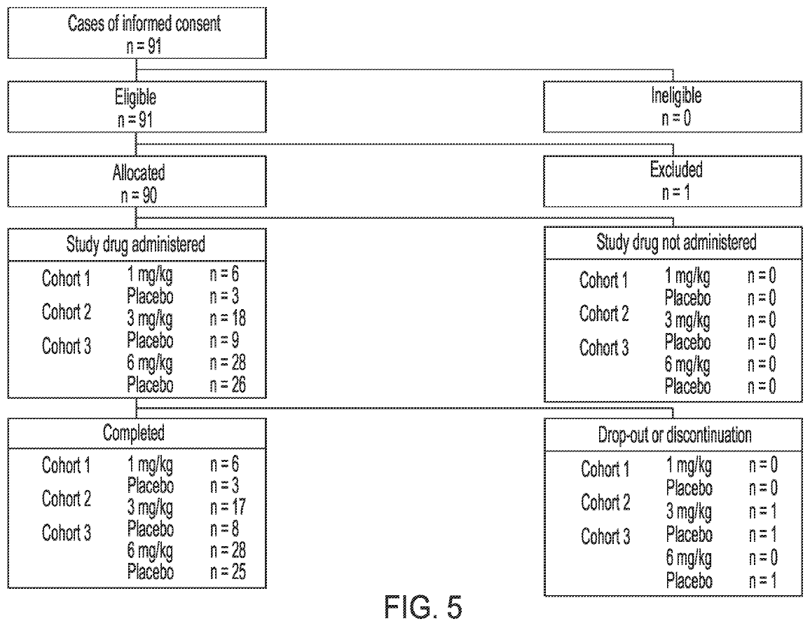

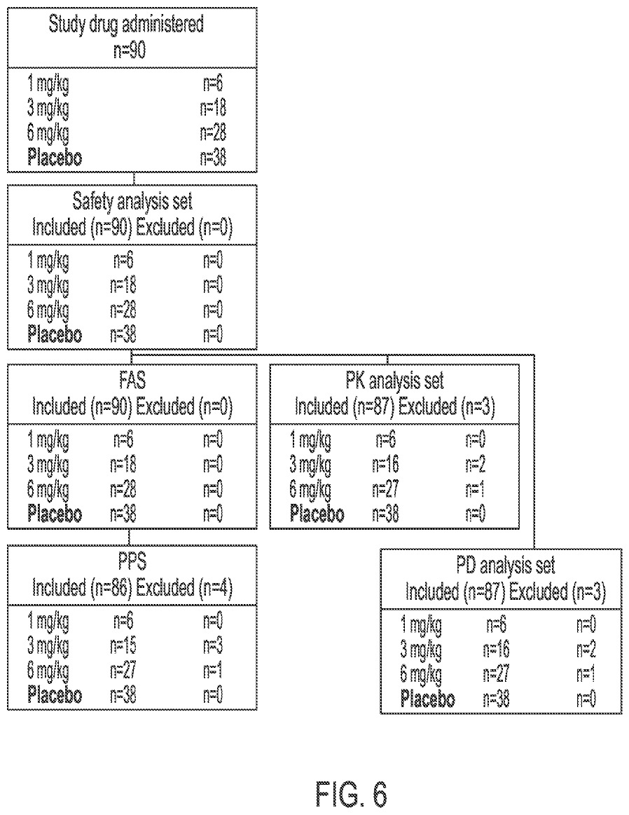

Details of the subjects are shown in FIG. 5 and details of analysis sets are shown in FIG. 6.

As for the background information of the safety analysis set, sex, presence or absence of a complication, cerebral infarction subtype (disease type), cerebrovascular recanalizing/blocked state, age, body weight, PT-INR value, NIHSS score, DWI-ASPECTS score, post-onset course hours, systolic blood pressure and diastolic blood pressure were analyzed; however, statistically significant difference was not found in TMS-007 (1, 3, 6 mg/kg) groups, a TMS-007-combined group, and a placebo group.

The average ages of subjects in the TMS-007 (1, 3, 6 mg/kg) groups, TMS-007-combined group and placebo group were 66.3 years old, 71.1 years old, 74.1 years old, 72.3 years old and 72.3 years old, respectively. The average times of them after onset were 9.16 hours, 9.21 hours, 9.76 hours, 9.50 hours and 9.33 hours, respectively. All subjects (90 cases) were given TMS-007 within 3-12 hours after onset, and then, 88 cases of the 90 cases were given TMS-007 within 4.5-12 hours.

### 2.1 Safety

With respect to the safety analysis set, symptomatic intracranial hemorrhage with NIHSS score worsening of 4 or more points was not observed in the TMS-007 groups but observed at a rate of 1/38 cases (2.6%) in the placebo group. The frequencies of occurrence of symptomatic intracranial hemorrhage with NIHSS score worsening of 4 or more points of individual TMS-007 groups, combined group and placebo group were compared by Fisher's exact test. As a result, all were not statistically significant. It was suggested that TMS-007 does not promote symptomatic intracranial hemorrhage with NIHSS score worsening of 4 or more points up to 24 hours after administration compared to the placebo group. Similar results were obtained in the PPS set.

In the safety analysis set, the frequency of occurrence of major hemorrhage up to Day 7 after administration was not observed in either the TMS-007 groups or the placebo group. The same results were obtained in the PPS set.

With respect to the safety analysis set, symptomatic intracranial hemorrhage within 24 hours after administration was not observed in the TMS-007 groups but observed at a rate of 2/38 cases (5.3%) in the placebo group. The frequencies of occurrence of symptomatic intracranial hemorrhage within 24 hours of individual TMS-007 groups, combined group and placebo group were compared by Fisher's exact test. As a result, all were not statistically significant. It was suggested that TMS-007 does not promote symptomatic intracranial hemorrhage within 24 hours after administration compared to the placebo group. Similar results were obtained in the PPS set.

With respect to the safety analysis set, overall mortality during the test period (up to Day 90) in the TMS-007 (1, 3, 6 mg/kg) groups and placebo group were 0/6 cases (0.0%), 1/18 cases (5.6%), 0/28 cases (0.0%) and 2/38 cases (5.3%), respectively. The overall mortality of the TMS-007 combined group was 1/52 cases (1.9%). The overall mortality of individual TMS-007 groups, combined group and placebo group were compared by Fisher's exact test. As a result, all were not statistically significant. It was suggested that TMS-007 does not promote overall mortality during the test period (up to Day 90) compared to the placebo group. Similar results were obtained in the PPS set.

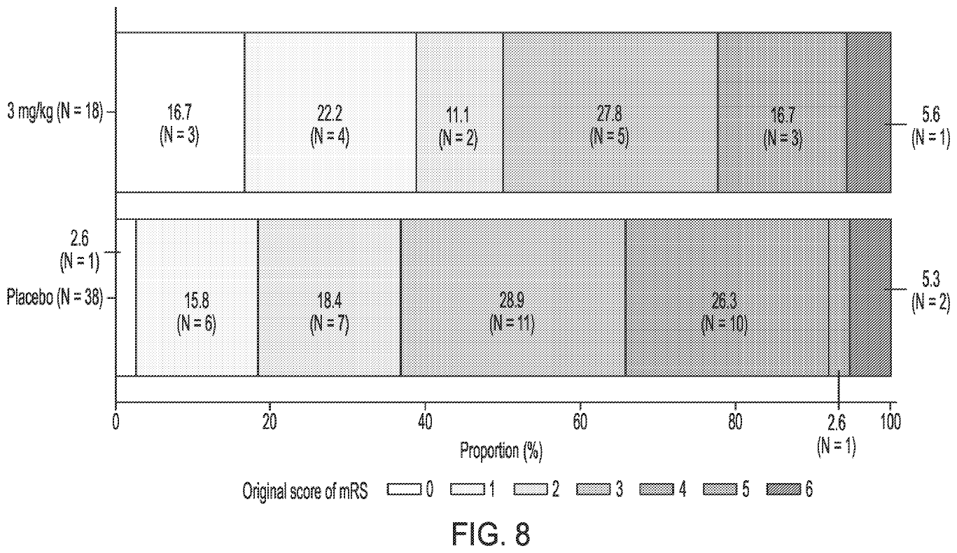

The frequencies of occurrence of an adverse event, serious adverse event, side effect, serious side effect, and adverse event leading to discontinuation of individual TMS-007 groups, combined group and placebo group during the test period (up to Day 90) were compared by Fisher's exact test. As a result, all were not statistically significant. Similar results were obtained in the PPS set (FIG. 8).

### 2.2 Effectiveness

(1) mRS

In the FAS group, the ratios of mRS 0-1 of TMS-007 (1, 3, 6 mg/kg) groups, combined group and placebo group were 3/6 cases (50.0%), 7/18 cases (38.9%), 11/28 cases (39.3%), 21/52 cases (40.4%), 7/38 cases (18.4%), respectively. Similar results were obtained in the PPS set. In FAS group, the ratios of mRS 0-2 of TMS-007 (1, 3, 6 mg/kg) groups, combined group and placebo group were 4/6 cases (66.7%), 9/18 cases (50.0%), 15/28 cases (53.6%), 28/52 cases (53.8%), 14/38 cases (36.8%), respectively.

In the FAS group, to mRS evaluation of individual dose groups (mRS 0-1 is assigned 1, mRS 2-6 is assigned 0) Day 90 after administration (Day 90), a logistic model using response, dose groups (TMS-007-combined group and placebo group), mRS on the base line, NIHSS score, age, blood pressure (systolic blood pressure) as explanatory variables was applied, odds ratio and 95% confidence interval of the TMS-007-combined group relative to the placebo group were estimated. The same estimation was carried out by using TMS-007 dose groups as the dose groups relative to the placebo group. More specifically, the odds ratios of individual TMS-007 dose groups relative to the placebo group and 95% confidence interval were estimated.

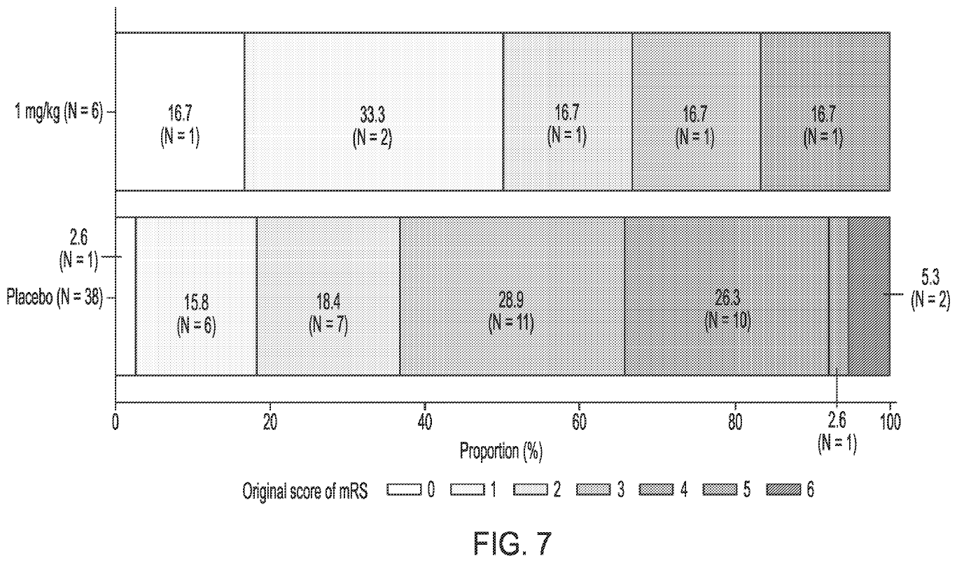

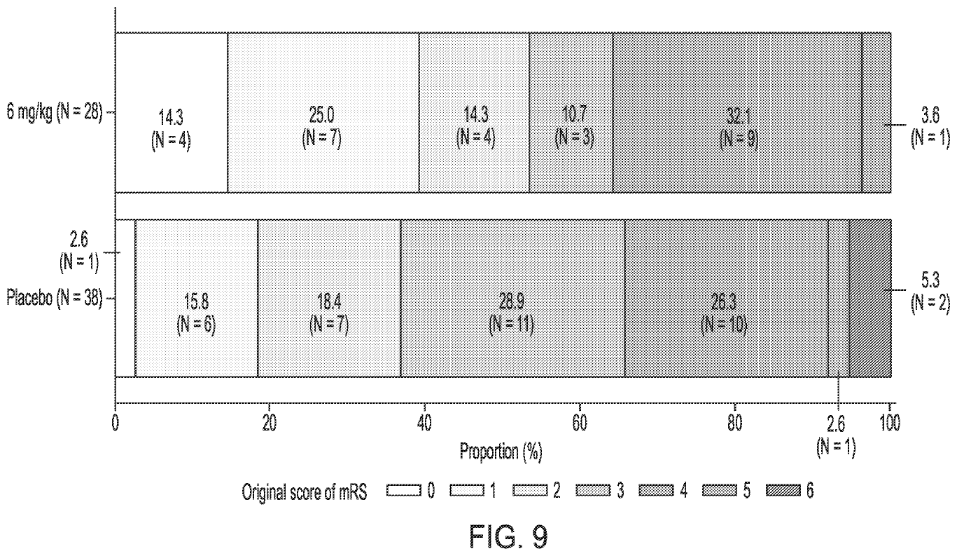

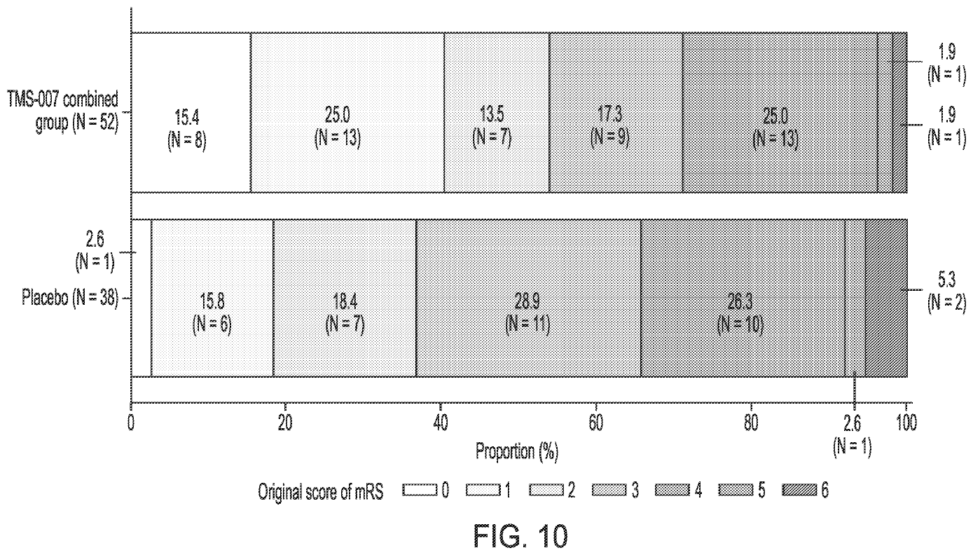

The results are shown in FIGS. 7 to 10 and Tables 2 to 4. The intercept estimation values of mRS were −6.193 and −6.434, respectively. The odds rate of mRS of the dose groups relative to mRS of the placebo group was 0.299 and the 95% confidence interval thereof was 0.099-0.902. It was suggested that mRS of the dose groups is statistically significantly lower (a therapeutic effect is present) than the placebo group. The odds ratios and 95% confidence interval (described within parentheses) of mRS of the TMS-007 groups relative to mRS of the placebo group were as follows: 0.088 (0.010-0.787) in TMS-007 (1 mg/kg) dose group, 0.484 (0.120-1.945) in TMS-007 (3 mg/kg) dose group and 0.299 (0.083-1.072) in the TMS-007 (6 mg/kg) dose group. Since these values were smaller than the odds ratio 1 of these dose groups relative to the placebo group, it was suggested that a therapeutic effect is present. Particularly, the value in a dose group (1 mg/kg) shows statistically significant difference. It was suggested that a therapeutic effect is present.

TABLE 2
Odds ratio of mRS of TMS-007 combined group to placebo group
on Day 90 after administration (mRS 0 and 1 is assigned
1, mRS 2, 3, 4, 5 and 6 are assigned 0) in the FAS set.

| End- | | Estimated | Standard | Odds | 95% confidence |
|---|---|---|---|---|---|
| point | | value | error | ratio | interval |
| mRS | Intercept | −6.193 | 2.931 | — | — |
| | Dose group | −1.206 | 0.563 | 0.299 | 0.099, 0.902 |

—: Out of range

TABLE 3
Odds ratio of mRS of individual dose groups to placebo group
on Day 90 after administration (mRS 0 and 1 is assigned
1, mRS 2, 3, 4, 5 and 6 are assigned 0) in the FAS set.

| End- | | Estimated | Standard | Odds | 95% confidence |
|---|---|---|---|---|---|
| point | | value | error | ratio | interval |
| mRS | Intercept | −6.434 | 3.029 | — | — |
| | 1 mg/kg | −2.425 | 1.115 | 0.088 | 0.010, 0.787 |
| | 3 mg/kg | −0.726 | 0.710 | 0.484 | 0.120, 1.945 |
| | 6 mg/kg | −1.207 | 0.652 | 0.299 | 0.083, 1.072 |

—: Out of range

Note that, the rates of obtaining outcome of mRS0-2 on Day 90 after administration (shown below) were values not showing statistically significant differences but consequently suggested effectiveness of TMS-007.

TABLE 4
Rate of obtaining outcome of mRS0-2
on Day 90 after administration.

| | | Odds | 95% confidence |
|---|---|---|---|
| Endpoint | | ratio | interval |
| mRS | Intercept | | |
| | 1 mg/kg | 0.292 | 0.047, 1.802 |
| | 3 mg/kg | 0.585 | 0.188, 1.815 |
| | 6 mg/kg | 0.506 | 0.187, 1.365 |
| | TMS-007 combined group | 0.500 | 0.213, 1.177 |

### (2) Recanalization Rate

The recanalization rate was evaluated by MRA. Patients (39 cases) observed to have occlusion at the time of enrollment and observed to be “canalized” or “partially canalized” on Day 2 were evaluated as recanalization cases. The ratio of the recanalization cases was 14/21 cases (58.3%) in the TMS-007 administration groups and 4/15 cases (26.7%) in the placebo group. From this, an improvement in the TMS-007 administration group was observed (odds ratio: 4.23, 95% confidence interval CI: 0.99, 18.07).

As mentioned above, it was confirmed that TMS-007 is safe and effective for patients with acute cerebral infarction within 12 hours after onset at a dose of 1-6 mg/kg.

### 2.3 Pharmacokinetics (PK)

In the PK analysis set, the dose groups except the placebo group were subjected to counting/analysis. The plasma TMS-007 concentrations (original scale value, mean±standard deviation) of the TMS-007 (1, 3, 6 mg/kg) groups (hereinafter described in the same order) 31 minutes after administration of a study drug were 7.294±1.472, 29.28±8.425 and 52.19±12.76 μg/mL, respectively; the plasma TMS-007 concentrations 2 hours after the administration were 0.1379±0.09777, 0.8970±0.5752 and 3.033±2.924 μg/mL, respectively. With respect to the plasma concentration value (logarithmic conversion value) of a study drug 31 minutes after initiation of administration, difference between doses was evaluated by one-way ANOVA using a dose as a factor. As a result, p value was less than 0.0001 and difference was statistically significant

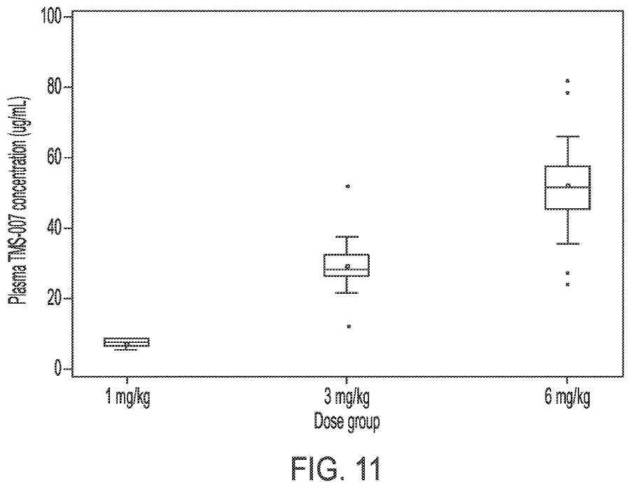

When the dose proportionality of plasma TMS-007 concentration of the investigational new drug 31 minutes after initiation of administration, was evaluated, a point estimation value (two-sided 95% confidence interval) of slope (b) of a regression equation of power model was 1.042 (0.895-1.189). From this, linearity of TMS-007 in the dose range of 1 to 6 mg/kg was confirmed (see, FIG. 11).

### 3. Consideration

A drug, t-PA, widely used as a cerebral infarction drug is considered to show clinical utility mainly due to thrombolytic effect. Other compounds such as desmoteplase and tenecteplase have been developed as a thrombolytic agent, are proteins similarly to t-PA. In fact, up to present, a lower molecular compound having an effect to remove thrombi of cerebral infarction patients has not been known (Mican et al., Computational and Structural Biotechnology Journal, Vol. 17, 2019, Pages 917-938; Nikitin et al., J Stroke. 2021; 23 (1): 12-36).

A drug t-PA is known to increase symptomatic intracranial hemorrhage of cerebral infarction patients. Other cerebral infarction drug candidate substances having a thrombolytic effect are known to facilitate intracranial hemorrhage (Wardlaw et al., Lancet. 2012 Jun. 23; 379 (9834): 2364-72). Likewise, it has been considered that t a risk of intracranial hemorrhage is increased by the thrombolytic effect (Shi et al., Sci Rep 6,33989 (2016); Kvistad et al., Stroke. Stroke. 2019 May; 50 (5): 1279-1281). However, the above results actually obtained in the clinical trial in cerebral infarction patients demonstrate that TMS-007 does not increase a frequency of occurrence of symptomatic intracranial hemorrhage compared to a placebo group.

### INDUSTRIAL APPLICABILITY

The present invention is useful for treatment of cerebral infarction, particularly treatment of cerebral infarction patients to whom conventional thrombolytic therapy and thrombectomy therapy is not applicable.

All publications, patents and patent applications cited in the specification are incorporated herein in their entireties by reference.

### CITATION LIST

### Patent Literatures

Patent Literatures 1: Japanese Patent Laid-Open No. 2004-224737
Patent Literatures 2: Japanese Patent Laid-Open No. 2004-224738
Patent Literatures 3: WO2007/111203
Patent Literatures 4: WO2011/004620

### Non Patent Literatures

Non Patent Literature 1: Takayasu et al., “Enhancement of fibrin binding and activation of plasminogen by staplabin through induction of a conformational change in plasminogen” FEBS Letter 1997; 418: 58-62
Non Patent Literature 2: Matsumoto et al., “Soluble Epoxide Hydrolase as an Anti-inflammatory Target of the Thrombolytic Stroke Drug SMTP-7” J Biol Chem 2014; 289: 35826-35838

1. A pharmaceutical composition for treating cerebral infarction, comprising Compound I having the structure of formula (I) or a salt thereof, which is administered to a subject at a dose of 1 to 6 mg/kg as said Compound I.

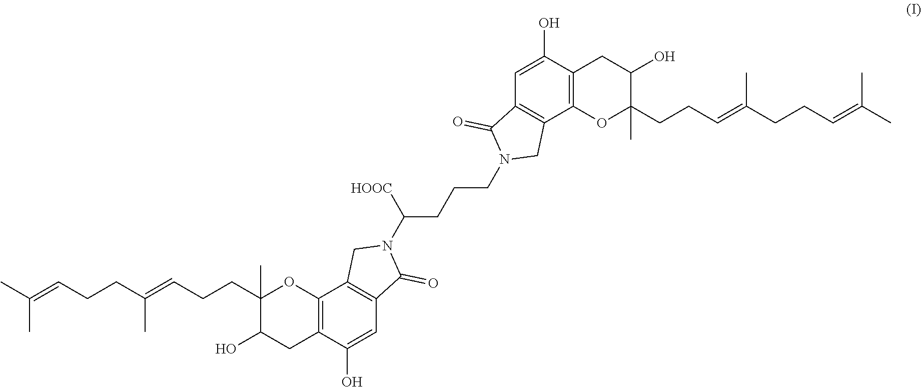 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

2. The pharmaceutical composition according to claim 1, which is administered to a subject at a dose of 1 mg/kg as said Compound I.

3. The pharmaceutical composition according to claim 1, which is administered to a subject at a dose of 3 mg/kg as said Compound I.

4. The pharmaceutical composition according to claim 1, which is administered to a subject at a dose of 6 mg/kg as said Compound I.

5. The pharmaceutical composition according to any one of claims 1 to 4, which is administered to a subject within 12 hours of the onset of cerebral infarction.

6. The pharmaceutical composition according to any one of claims 1 to 4, which is administered to a subject 3 to 12 hours after the onset of cerebral infarction.

7. The pharmaceutical composition according to any one of claims 1 to 4, which is administered to a subject 4.5 to 12 hours after the onset of cerebral infarction.

8. The pharmaceutical composition according to any one of claims 1 to 7, which is administered to a subject having a risk of causing hemorrhage.

9. The pharmaceutical composition according to claim 8, wherein the hemorrhage is intracranial or extracranial hemorrhage.

10. The pharmaceutical composition according to any one of claims 1 to 9, which is administered to a subject with cerebral infarction having vascular occlusion including partial occlusion.

11. The pharmaceutical composition according to any one of claims 1 to 10, which is administered intravenously.

12. The pharmaceutical composition according to any one of claims 1 to 11, which is administered once daily, preferably in a single dose.

13. The pharmaceutical composition according to any one of claims 1 to 12, which is administered as a bolus followed by continuous administration.

14. The pharmaceutical composition according to claim 13, which is administered intravenously over 1 minute followed by infusion over 30 minutes.

15. The pharmaceutical composition according to any one of claims 1 to 14, which can be administered to a subject to whom administration of a thrombolytic agent or physical thrombectomy is difficult or impossible.

16. The pharmaceutical composition according to claim 15, wherein the thrombolytic agent is a tissue plasminogen activator.

17. The pharmaceutical composition according to any one of claims 1 to 16, wherein the cerebral infarction is atherothrombotic/embolic cerebral infarction, cardiogenic cerebral infarction or lacunar cerebral infarction.

18. The pharmaceutical composition according to any one of claims 1 to 17, wherein a single dose of Compound I or a salt thereof is contained in a sealed container together with a pharmacologically acceptable carrier.

19. The pharmaceutical composition according to any one of claims 1 to 18, wherein 20 mg to 1000 mg of Compound I or a salt thereof is contained in a sealed container together with a pharmacologically acceptable carrier.

20. A pharmaceutical composition for treating cerebral infarction, comprising Compound I having the structure of formula (I) or a salt thereof, which is administered to a subject within 12 hours of the onset of cerebral infarction.

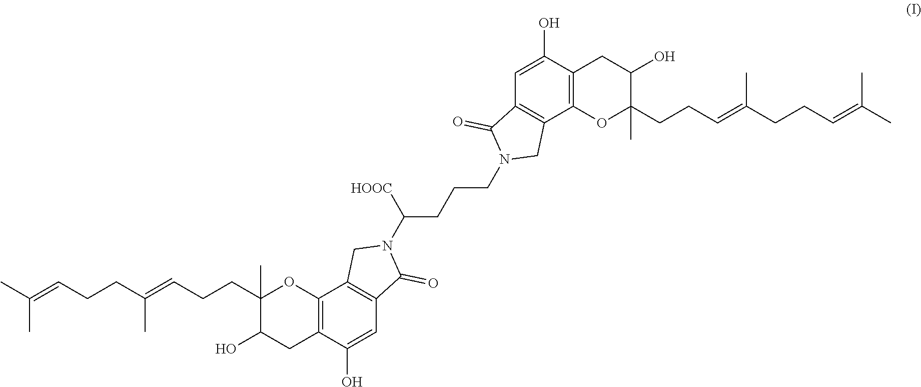 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

21. The pharmaceutical composition according to claim 20, which is administered to a subject 3 to 12 hours after the onset of cerebral infarction.

22. The pharmaceutical composition according to claim 20, which is administered to a subject 4.5 to 12 hours after the onset of cerebral infarction.

23. The pharmaceutical composition according to any one of claims 20 to 22, wherein the composition is administered to a subject at a dose of 1 to 6 mg/kg as said Compound I.

24. The pharmaceutical composition according to any one of claims 20 to 22, wherein the composition is administered to a subject at a dose of 1 mg/kg as said Compound I.

25. The pharmaceutical composition according to any one of claims 20 to 22, wherein the composition is administered to a subject at a dose of 3 mg/kg as said Compound I.

26. The pharmaceutical composition according to any one of claims 20 to 22, wherein the composition is administered to a subject at a dose of 6 mg/kg as said Compound I.

27. The pharmaceutical composition according to any one of claims 20 to 26, which is administered to a subject having a risk of causing hemorrhage.

28. The pharmaceutical composition according to claim 27, wherein the hemorrhage is intracranial or extracranial hemorrhage.

29. The pharmaceutical composition according to any one of claims 20 to 28, which is administered to a subject with cerebral infarction having vascular occlusion including partial occlusion.

30. The pharmaceutical composition according to any one of claims 20 to 29, which is administered intravenously.

31. The pharmaceutical composition according to any one of claims 20 to 30, which is administered once daily, preferably in a single dose.

32. The pharmaceutical composition according to any one of claims 20 to 31, which is administered as a bolus followed by continuous administration.

33. The pharmaceutical composition according to claim 32, which is administered intravenously over 1 minute followed by infusion over 30 minutes.

34. The pharmaceutical composition according to any one of claims 20 to 33, which can be administered to a subject to whom administration of a thrombolytic agent or physical thrombectomy is difficult or impossible.

35. The pharmaceutical composition according to claim 34, wherein the thrombolytic agent is a tissue plasminogen activator.

36. The pharmaceutical composition according to any one of claims 20 to 35, wherein the cerebral infarction is atherothrombotic/embolic cerebral infarction, cardiogenic cerebral infarction or lacunar cerebral infarction.

37. A pharmaceutical composition for treating cerebral infarction, comprising Compound I having the structure of formula (I) or a salt thereof, wherein the composition is to recanalize occluded vessel in cerebral infarction.

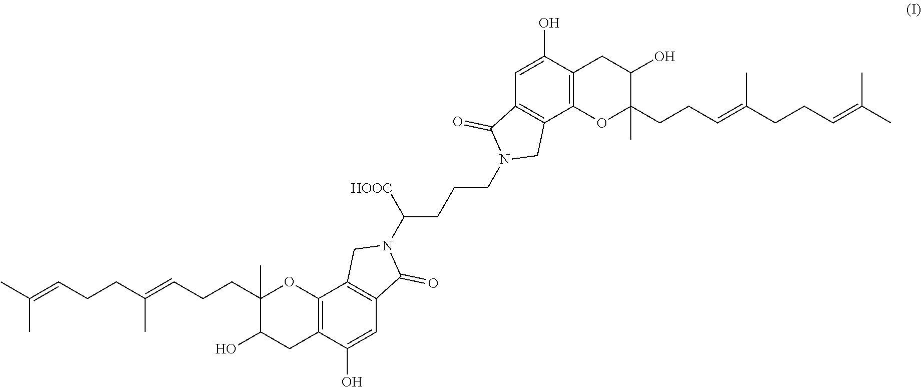 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

38. A pharmaceutical composition for treating cerebral infarction, comprising Compound I having the structure of formula (I) or a salt thereof, wherein the composition is administered to a subject having a risk of causing hemorrhage.

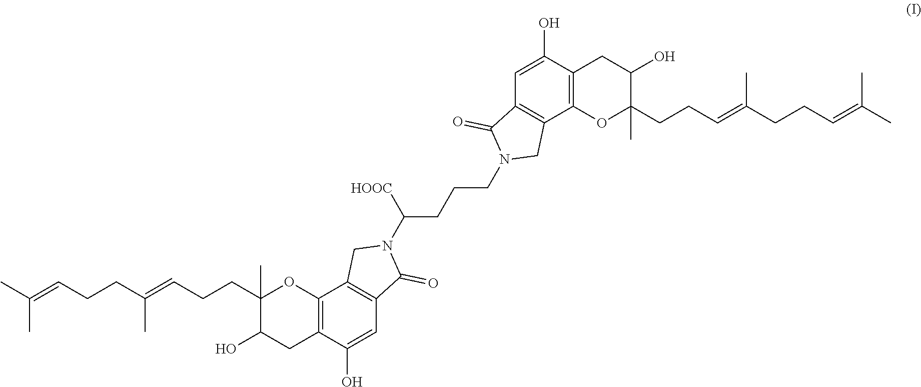 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

39. A pharmaceutical composition for reducing risk of causing hemorrhage in cerebral infarction, comprising Compound I having the structure of formula (I) or a salt thereof.

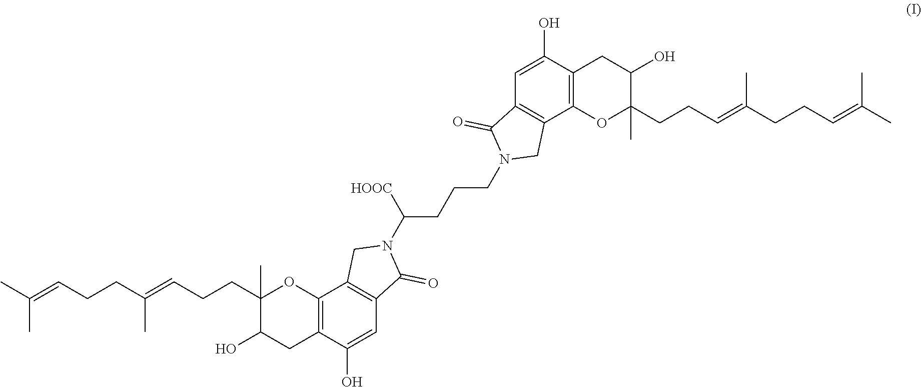 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

40. A pharmaceutical composition for treating cerebral infarction, comprising Compound I having the structure of formula (I) or a salt thereof, wherein the composition is to improve a life of independence after cerebral infarction.

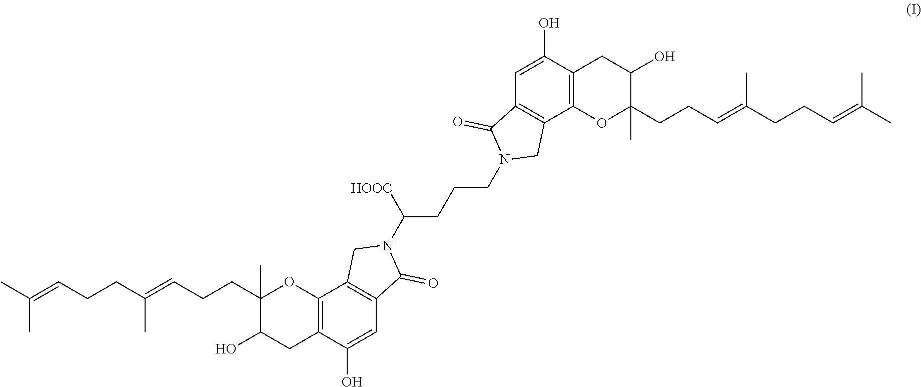 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

41. The pharmaceutical composition according to claim 32, wherein an index of the improvement of a life of independence is outcome of mRS (modified Rankin Scale) of 0-1 on Day 90 after administration.

42. The pharmaceutical composition according to claim 40, wherein an index of the improvement of a life of independence is outcome of mRS (modified Rankin Scale) of 0-2 on Day 90 after administration.

43. A pharmaceutical composition for treating cerebral infarction, comprising Compound I having the structure of formula (I) or a salt thereof, wherein the composition has an action to dissolve a thrombus of a cerebral infarction patient.

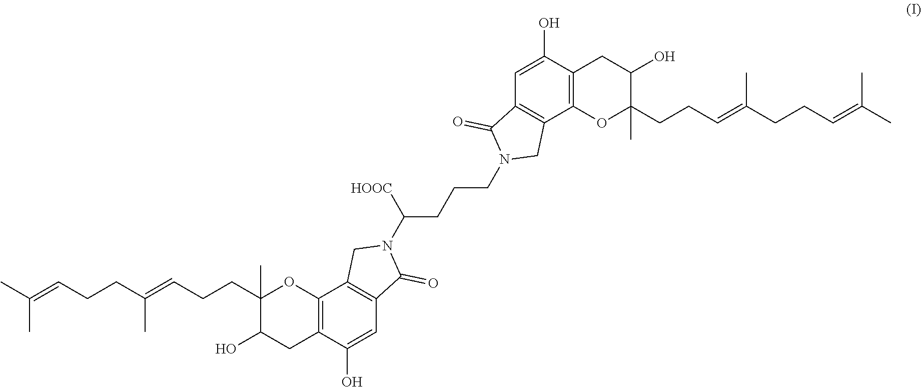 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

44. A pharmaceutical composition for treating cerebral infarction, comprising Compound I having the structure of formula (I) or a salt thereof, wherein the composition has a low risk of a hemorrhagic side effect.

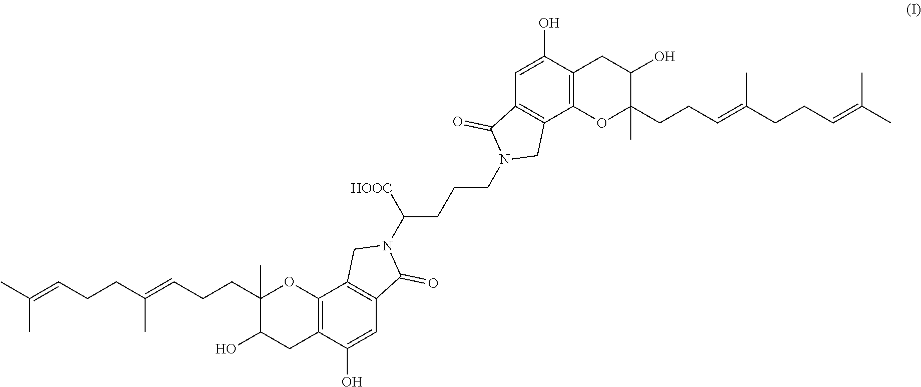 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

45. The pharmaceutical composition according to claim 44, wherein the hemorrhagic side effect is symptomatic intracranial hemorrhage.

46. The pharmaceutical composition according to any one of claims 1 to 45, comprising either or both of a basic additive and an amphipathic additive.

47. The pharmaceutical composition according to claim 46, wherein the basic additive is one or more selected from the group consisting of amino sugars, alkanolamines and trometamol salts, preferably, one or more selected from the group consisting of meglumine, triethanolamine and trometamol hydrochloride, and wherein the amphipathic additive is one or more selected from the group consisting of polyoxyethylene castor oil, polyoxyethylene hydrogenated castor oil, polyoxyethylene sorbitan monolaurate, polyoxyethylene-polyoxypropylene glycol, polysorbate, polyethylene glycol, ursodesocycholic acid, sorbitan fatty acid ester and sodium desoxycholate, preferably, one or more selected from the group consisting of polyoxyethylene castor oil, polyoxyethylene hydrogenated castor oil and polyoxyethylene sorbitan monolaurate.

48. The pharmaceutical composition according to any one of claims 1 to 47, wherein Compound I is SMTP-7 represented by formula (II):

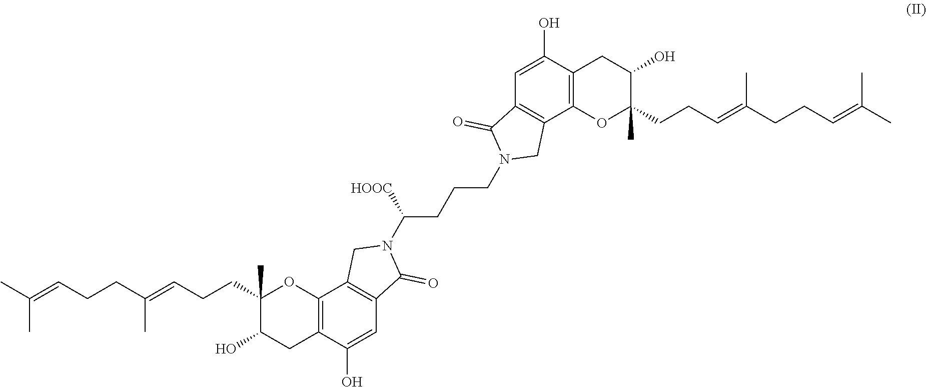 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

49. The pharmaceutical composition according to any one of claims 1 to 48, wherein cerebral infarction is ischemic stroke.

50. The pharmaceutical composition according to claim 49, wherein the cerebral infarction is acute cerebral infarction or wherein the ischemic stroke is acute ischemic stroke.
